(19) United States
(12) Patent Application Publication
(10) Pub. No.: US 2023/0218843 A1
(43) Pub. Date: Jul. 13, 2023

(54) MEDICAL VENTILATOR

(71) Applicant: RFDesign Pty Ltd, Red Hill, Brisbane, QLD (AU)

(72) Inventor: Seppo SAARIO, Red Hill, QLD (AU)

(21) Appl. No.: 18/001,563

(22) PCT Filed: Jun. 14, 2021
(86) PCT No.: PCT/AU2021/050608

§ 371 Date: Dec. 12, 2022

(30) Foreign Application Priority Data
Jun. 12, 2020 (AU) 2020901942

(51) Int. Cl. A61M 16/00; A61M 16/20

(52) U.S. Cl. CPC A61M 16/0093; A61M 16/024; A61M 16/0066; A61M 16/209; A61M 2016/0027

(57) ABSTRACT

A ventilator including a housing; a gas inlet port disposed in the housing and adapted to be coupled to a gas source to receive a flow of gas; a valve assembly coupled with the gas inlet port for controlling flow of gas from the gas inlet port to a gas outlet port disposed in the housing and adapted for being coupled to a patient interface to fluidly couple the gas outlet port to the airway of a patient; a controller module disposed in the housing, the controller module comprising a controller operatively coupled with the valve assembly to control operation of the valve assembly; an airway pressure sensor positioned between the valve assembly and the patient interface to measure air flow output into flowing into the airway of the patient; wherein the pressure sensor is operatively connected to the controller module to control the operation of the valve assembly in response to changes in air flow output measured by the airway pressure sensor during use.

### TECHNICAL FIELD

The present invention relates to the field of medical ventilators and more specifically to a portable ventilator.

### BACKGROUND

Any references to methods, apparatus or documents of the prior art are not to be taken as constituting any evidence or admission that they formed, or form part of the common general knowledge.

Ventilators for patients requiring breathing assistance have traditionally been large, heavy, power-hungry devices that have provided little if any mobility to a patient. In the coronavirus pandemic, ventilators help patients breathe and have been highly sought after and hard to find. According to the World Health Organisation, one in six COVID-19 patients becomes seriously ill and has difficulty breathing. The patient's lungs appear to be acutely affected in this particular pandemic and it has become known that COVID-19 infections can cripple breathing functions.

Medical ventilators gently pump air through a breathing tube into the patient's lungs and allow the patient to exhale. The use of the ventilator gives patients oxygen and removes carbon dioxide, which, if not expelled, can damage the patient's organs. In some cases, air with higher oxygen content may also need to be used.

During the recent pandemic, health officials have warned of a critical shortage of ventilators as these infections continue to rise through various communities around the world it has become clear that expensive stand-alone ventilators are not particularly suitable for large scale use during a pandemic and it is desirable to provide a ventilator which has a relatively smaller footprint that is easily deployable on a large scale in a pandemic situation.

### SUMMARY OF INVENTION

In an aspect, the invention provides a ventilator comprising:
a housing;
a gas inlet port disposed in the housing and adapted to be coupled to a gas source to receive a flow of gas;
a valve assembly coupled with the gas inlet port for controlling flow of gas from the gas inlet port to a gas outlet port disposed in the housing and adapted for being coupled to a patient interface to fluidly couple the gas outlet port to the airway of a patient;
a controller module comprising a controller operatively coupled with the valve assembly to control operation of the valve assembly;
an airway pressure sensor positioned between the valve assembly and the patient interface to measure air flow output flowing into the airway of the patient;
   wherein the pressure sensor is operatively connected to said controller module to control the operation of the valve assembly in response to changes in air flow output measured by the airway pressure sensor during use.

In an embodiment, the ventilator further comprises: an inlet pressure sensor disposed between the gas inlet port and the valve assembly for measuring gas flow input flowing into the gas inlet port wherein the inlet pressure sensor is operatively connected to the controller module to control the operation of the valve assembly in response to changes in air flow input measured by the inlet pressure sensor during use.

In an embodiment, the ventilator further comprises an oxygen inlet port adapted to be coupled to an oxygen gas source for allowing oxygen gas to flow from the cylinder to the outlet port.

In an embodiment, the ventilator further comprises an oxygen pressure sensor for measuring flow rate of oxygen flowing into the outlet port for being conveyed to the airway of the patient.

In an embodiment, the oxygen pressure sensor is operatively connected to the controller module to control the operation of an oxygen regulating module in response to changes in air flow input or air flow output measured by the inlet pressure sensor or the airway pressure sensor during use.

In an embodiment, the ventilator further comprises: a relief valve disposed in the housing and adapted to be coupled with the gas outlet port for releasing gas from said relief valve when pressure of gas flowing from the valve assembly to the outlet exceeds a pre-set pressure value.

In an embodiment, the valve assembly comprises a rotary valve assembly further comprising:
a valve body defining at least one input port and at least one output port, each port providing a separate fluid communication path between an outer surface of the valve body;
a bore extending along a longitudinal axis defined by the valve body, and
a valve gate rotatably positioned in the bore to rotate between an open position and a closed position wherein in the open position the gas is conveyed from the inlet port to the outlet port.

In an embodiment, the valve gate comprises a plurality a plurality of flow passages for conveying fluid from the inlet port to the outlet port wherein rotation of the valve gate determines the flow rate of the gas from the inlet port to the outlet port.

In an embodiment, the valve gate is driven by a servo motor, the servo motor being operatively coupled to the controller module for controlling the rotation of the valve gate in response to changes in air flow output measured by the airway pressure sensor and/or changes in air flow input measured by the inlet pressure sensor.

In an embodiment, the ventilator further comprises an ambient pressure sensor for sensing ambient pressure in the vicinity of the housing, the ambient pressure being operatively coupled with the controller module to control the operation of the valve assembly in response to changes in air flow input measured by the inlet pressure sensor during use.

In an embodiment, the controller module comprises a microprocessor arranged to be in communication with a peripheral interface wherein the peripheral interface is adapted to be connected to the airway pressure sensor.

In an embodiment, the peripheral interface is adapted to be connected to the inlet pressure sensor.

In an embodiment, the peripheral interface is adapted to be connected to the oxygen sensor.

In an embodiment, the microprocessor is in communication with a memory device, storing executable instructions wherein the processor when executing said instructions to monitor one or more of said inputs received on the peripheral interface for checking if one or more predetermined criteria have been satisfied and generating control signals for operating the valve assembly in response to checking if the said one or more predetermined criteria have been satisfied.

In an embodiment, the microprocessor is in communication with a transmitting module for transmitting one or more signals from the microprocessor, over a network, to one or more computing devices.

In an embodiment, the processor is in communication with a transceiver for receiving signals associated with instructions for operating the ventilator.

In an embodiment, the processor is in communication with a transceiver for reporting measurements of flow, respiratory rate, compliance and other operational information from the ventilator.

In an embodiment, the inlet port is adapted to be coupled with a source of compressed air generated by a compressor in a first operable configuration, said source of compressed air being fluidly coupled with a plurality of said inlet ports associated with corresponding ventilators simultaneously said first operating configuration.

In an embodiment, the inlet port is adapted to be coupled with a portable blower device for receiving a supply of gas from the blower.

In an embodiment, the inlet port is adapted to be coupled with a compressed air cylinder to provide the main source of gas for operating in a portable use case.

In another aspect, the invention provides a system comprising: a plurality of the ventilators in accordance with any one of the preceding claims wherein the inlet port for each of the plurality of ventilators is fluidly coupled to a common gas source to receive the flow of gas and wherein the outlet port for each of the plurality of ventilators is fluidly coupled to a common exhaust.

In an embodiment, the system further comprising an inlet gas regulator positioned in line with the common gas source for regulating the flow of gas to each of the inlet ports fluidly coupled to the common gas source.

In an embodiment the information flow from the ventilator unit can be routed to a remote display and control terminal where a plurality of ventilators can be controlled and monitored.

Automated alarms and limits for patient breathing performance may be used to alert doctors and medical professionals that may be located either locally or remotely.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a schematic illustration of a system 500 utilising a plurality of the medical ventilators 100.

FIG. 2 is a schematic illustration of a medical ventilator 100 in accordance with an embodiment.

FIG. 3 is a detailed schematic illustration of the controller 150 that forms part of the medical ventilator 100.

FIGS. 4A to 4D illustrate various detailed views of the valve assembly 150.

### DETAILED DESCRIPTION OF PREFERRED EMBODIMENTS

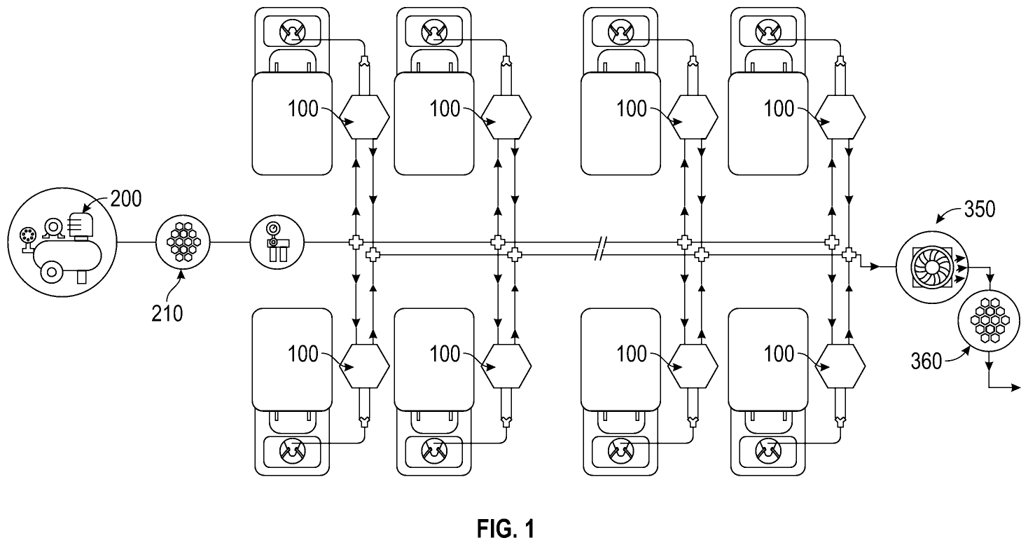

FIGS. 1 to 3 illustrate a portable ventilator 100 that is particularly well suited for large scale deployment in a pandemic scenario where hundreds of patients may simultaneously need ventilator support to receive assistance with breathing. It must be understood that the illustration shown in FIGS. 1 to 3 refer to a non-limiting embodiment of the presently described invention.

Directional phrases used herein, such as, for example, left, right, clockwise, counterclockwise, top, bottom, up, down, and derivatives thereof, relate to the orientation of the elements shown in the drawings and are not limiting upon the claims unless expressly recited therein. As employed herein, the term “number” shall mean one or more than one and the singular form of “a”, “an”, and “the” include plural referents unless the context clearly indicates otherwise. As employed herein, the statement that two or more parts are “connected” or “coupled” together shall mean that the parts are joined together either directly or joined together through one or more intermediate parts. Further, as employed herein, the statement that two or more parts are “attached” together shall mean that the parts are joined together directly.

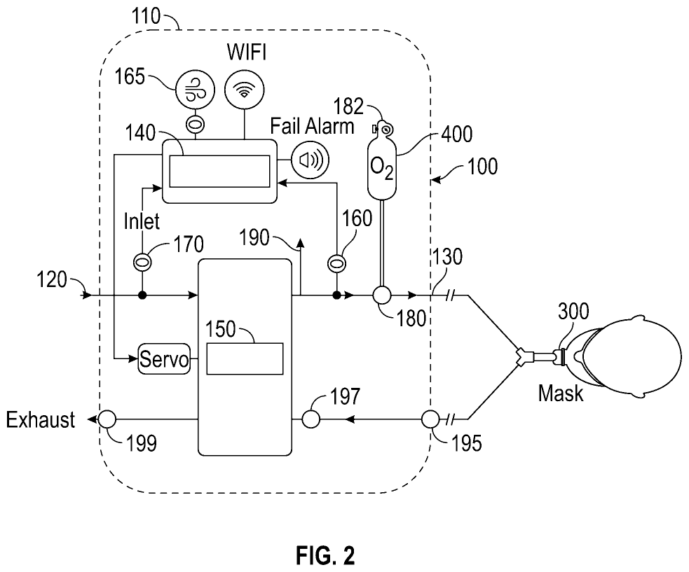

Referring to FIGS. 1 and 2, the portable ventilator 100 comprises a housing 110 with a gas inlet port 120 disposed in the housing. As shown in FIG. 1, gas inlet ports 120 for each of a plurality of the portable ventilators 100 may be connected to a common gas source 200 to receive a flow of gas. In the preferred embodiment, the gas source 200 may take the form of an air compressor that supplies compressed air to the gas inlet ports 120 for each of the portable ventilators 100 connected to the gas source 200. In such a configuration in which multiple portable ventilators 100, specifically gas inlet ports 120 of these ventilators 100 are connected to single source of gas 200 (such as an air compressor). In addition to the gas source 200, an inlet filter 210 and a regulator 220 may be used in line with the gas source 200. In some other embodiments, the gas inlet port 120 of a ventilator unit 100 may be coupled to an air blower particularly if one or more of the ventilator units 100 are operating in a stand-alone configuration.

Each of the ventilators 100 also comprises a gas outlet port 130 disposed in the housing 110. The gas outlet port 130 is adapted for being coupled to a patient interface such as a mask or an endotracheal tube (denoted generally by 300) to fluidly couple the gas outlet port 130 to the airway of a patient receiving respiratory support from the ventilator 100. Each portable ventilator 100 also includes a valve assembly 150 that is coupled with the gas inlet port 120 and the gas outlet port 130 for controlling the flow of gas being provided to the patient via the mask or endotracheal tube 300. The working of the valve assembly 150 will be explained in further detail in the foregoing sections. The portable ventilator 100 also includes a controller module 140 disposed in the housing 110 that is operatively coupled with the valve assembly 150 to control operation of the valve assembly 150 in response to feedback received from a plurality of pressure sensors or transducers. Specifically, at least a first airway pressure sensor 160 is positioned in between the valve assembly 150 and the mask or endotracheal tube 300 to measure air flow output flowing into the airway of the patient. The airway pressure sensor 160 is arranged within the housing of the ventilator 100 for measuring airway proximal pressure. Although airway and alveolar pressures are not instantly equal during positive-pressure ventilation, it is expected that proximal mean airway pressure (Paw) is a simple way of indirectly measuring mean alveolar pressure in the patient. As previously mentioned, the airway pressure sensor 160 is coupled with the controller module 140 for controlling operation of the valve assembly 150 in response to any changes in pressure values detected by the airway pressure sensor 160.

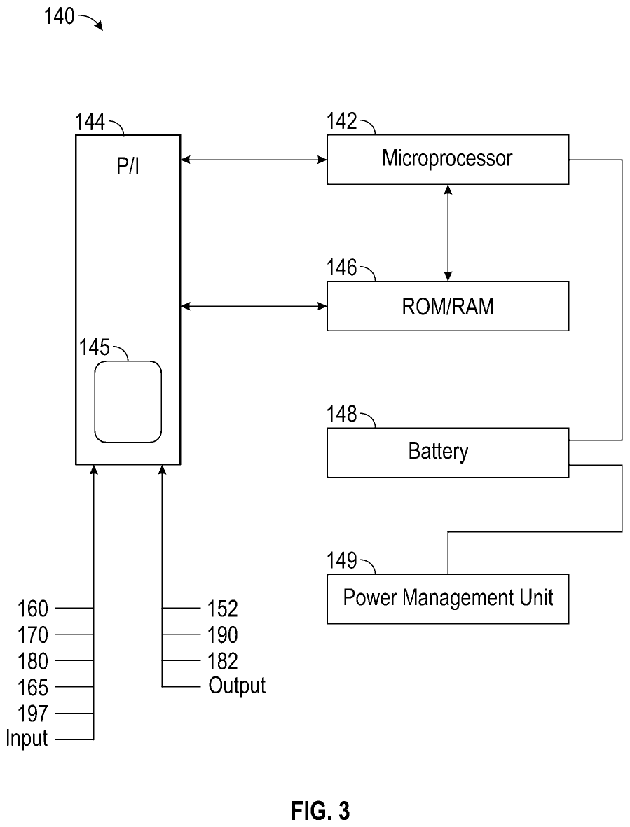

Referring to FIG. 3 in particular, in the preferred embodiment, the controller module 140 may include a microprocessor 142 arranged to be in communication with a peripheral interface 144 wherein the peripheral interface is adapted to be connected to the airway pressure sensor 160. In one example, the controller module 140 may take the form of a low-cost, low-power system on chip microcontroller module such as the ESP32 which employs a Tensilica Xtensa LX6 microprocessor that can be available in both dual-core and single core variations. The peripheral interface 144 of the microcontroller 142 may be arranged to receive input from the airway pressure sensor 160 and process the signals received from the pressure sensor 160 in accordance with one or more executable instructions saved on a memory device 146 such as a “Read-Only Memory” (ROM) device or a “Random Access Memory” (RAM) device communicating with the microprocessor 142. The pressure sensor 160 may take the form of an integrated silicon pressor sensor the MPX5010 which uses piezoresistive transducers and can be used with microcontroller such as the ESP32 by employing A/D inputs.

The valve assembly 150 may include a servo motor 152 coupled with a rotary valve 154 that controls the flow of gas from the inlet port 120 to the outlet port 140 in response to pressure variations detected by the pressure sensor 160. The use of a real time flow rate-indicative signal, generated by a flow transducer in the airway pressure sensor 160, as a feedback signal, wherein the instantaneously sensed flow rate is the parameter whose value is compared with the stored nominal value to generate the control signal for operating the servo motor 152 is helpful in achieving a closed-loop feedback system. This feedback system includes the provision of a servo motor 152 under the command of the microprocessor 142, to operate the rotary valve 154 and allows the ventilator 100 to achieve a relatively high degree of precision over a wide range of flow rates, with the ability to accommodate a wide variety of flow rate patterns.

In addition to the airway pressure sensor 160, the ventilator 100 also includes an inlet pressure sensor 170 disposed between the gas inlet port 120 and the valve assembly 150 for measuring gas flow input flowing into the gas inlet port 120. The inlet pressure sensor 170 may also take the form of a piezoresistive sensor such as the integrated silicon pressor sensor the MPX5010 that has been previously discussed. In addition to the airway pressure sensor, the inlet pressure sensor 170 may also be operatively connected to the controller module 140 to collectively control the operation of the valve assembly 150 in response to changes in air flow input measured by the inlet pressure sensor during use in combination with changes measured in airway pressure using the airway pressure sensor 160.

The ventilator 100 unit also includes an ambient pressure sensor 165 for sensing for sensing pressure values for ambient air in the vicinity of the patient. In some instances particularly in built environments (for example positive pressure medical rooms), the pressure around the patient may vary and in such instances any such variations are instantly sensed by the ambient pressure sensor 165. The ambient pressure sensor 165 is operatively coupled with the peripheral interface 144 of the EPS32 controller module 140 and data associated with the signals may be processed by the microprocessor 142 of the control module to generate control signals to control the operation of the servo motor 152 thereby controlling the operation of the valve assembly 150 in response to any changes measured by the ambient pressure sensor 165.

The ventilator 100 includes an oxygen inlet port 180 configured to be coupled to an oxygen gas source 400 for allowing oxygen gas to flow from the cylinder to the outlet port 130 to provide supplemental oxygen support to the patient's airway via the mask or endo-tracheal tube. A variably actuable valve 182 may be connected in series with the oxygen gas source 400 and the oxygen inlet port 180 to vary the fraction of positive pressure inspired oxygen (FiO₂) to the patient. FiO₂ will vary between 0.21, in which no supplemental oxygen support is provided to the patient, and 1.0, in which pure oxygen is provided to the patient. In order to determine the proper FiO₂, the arterial oxygen saturation (SpO₂) may typically be monitored via a pulse oximeter 184 attached to the patient. The SpO₂ is ideally in the range of 0.97-1.0 whereas an SpO₂ of less than 0.91 is dangerously low. Consequently, the FiO₂ should be increased as the SpO₂ decreases.

In at least some embodiments, the pulse oximeter may communicate directly with the controller module 140 via a trans-receiver 145 arranged in communication with the microprocessor 142 of the ESP32 microcontroller. The memory device 146 may include executable instructions to generate control signals for controlling the variably actuable valve 182 and vary the flow of oxygen flowing into the outlet port 130 in response to changes in the arterial oxygen saturation. In the preferred embodiment, a small oxygen cylinder may be used as an oxygen source 400 for each individual ventilator 100 as shown in FIGS. 1 and 2. This portable oxygen cylinder source 400 in at least some instances may be replaced by a centralised oxygen distribution manifold. Once again, it is important to appreciate that in at least some embodiments, control of the variably actuable valve 182 for controlling the flow of oxygen to the outlet port 130 is carried out simultaneously whilst also controlling the operation of the valve assembly 150. In this regard, the valve assembly 150 may include a plurality of valves including the previously mentioned rotary valve 154 (actuated by the servo motor 152) and the variably actuable valve 182 that separately controls the flow of oxygen in response to oxygen levels sensed by the oxygen sensor in the form of the pulse oximeter 184.

The ventilator 100 comprises a built-in safety mechanism for avoiding any instances where gas flowing through the outlet 130 into the airway of the patient is supplied at dangerously high pressure values. An excessively high respiration pressure may have various causes. For example, the breathing gas flow controlled by the valve assembly 150 may be set to an excessively high value due to malfunction or error or the inhalation tube leading to the patient has inadvertently become kinked. In the presently described ventilator 100, an overpressure valve 190 and a discharge port 192 is connected in series with the inhalation line that allows flow of gas from an outlet the valve assembly 150 to the outlet port 130. In some embodiments, the overpressure valve 190 may take the form of a mechanical valve with a set pressure limitation such that once the pressure in the inhalation line exceeds a preset limit, the overpressure valve releases the pressure in the inhalation line by opening the discharge port 192. In other embodiments, the pressure relief valve or overpressure valve 190 may be provided in the form of an electronic relief valve which can be re-used when the overpressure event has ended or when the pressure settings have been reset by the user. The electronic pressure relief valve 190 may be arranged in communication with the peripheral interface 144 of the ESP32 microcontroller module 140.

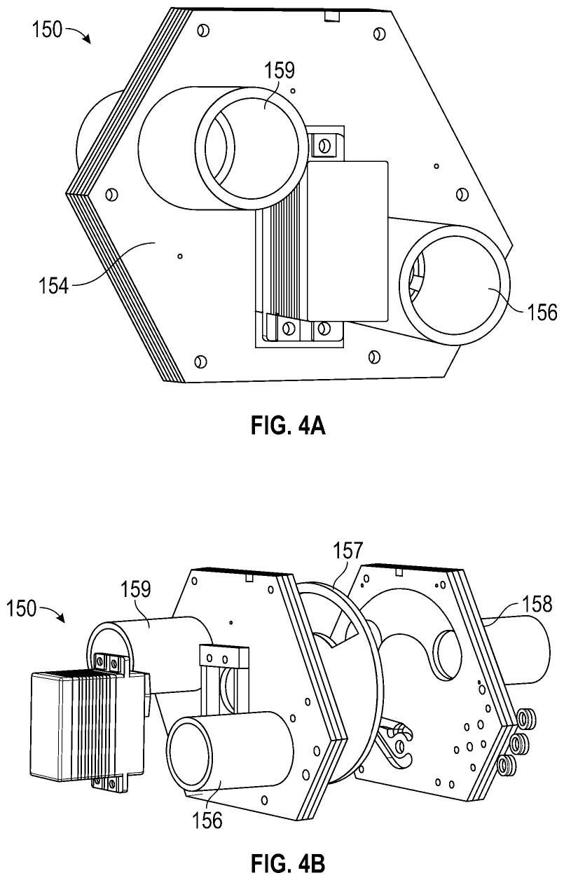

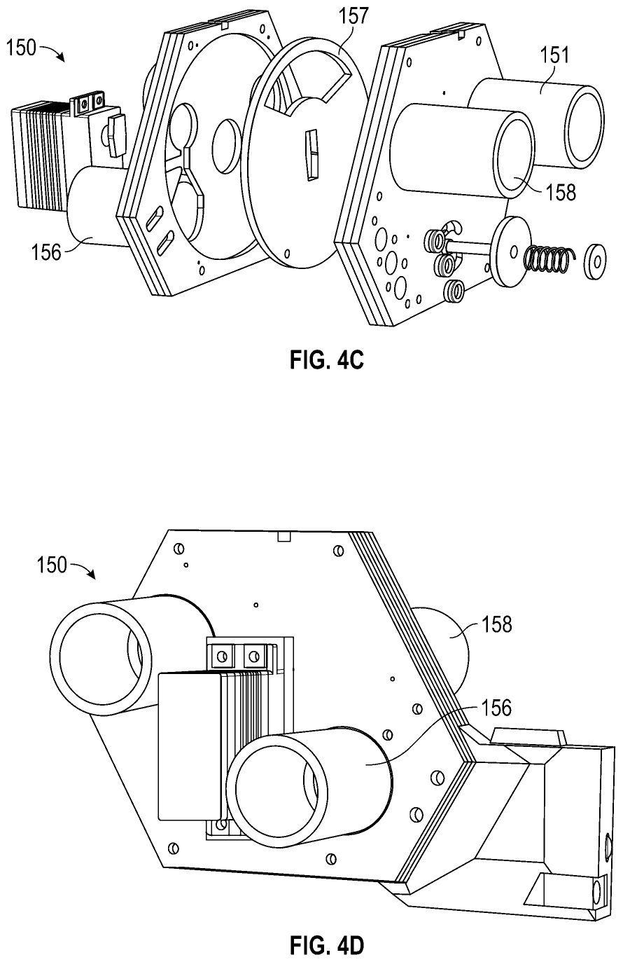

Turning to the valve assembly 150 shown in FIG. 4, in the preferred embodiment, the valve assembly 150 may take the form of rotary valve assembly comprising a valve body 154 defining at least one input port 156 and at least one output port 158. Each of the input port 156 and the output port 158 provide a separate fluid communication path between an outer portion of the valve body 154 and the gas inlet port 120 and the gas outlet port 130 respectively. A bore 155 extends along a longitudinal axis defined by the valve body 154 and a valve gate 157 is rotatably positioned in the bore to rotate between an open position and a closed position wherein in the open position the gas is conveyed from the inlet port 120 to the outlet port 130. The rotatable valve gate 157 may include a plurality of flow passages for conveying fluid from the inlet port 120 to the outlet port 130 wherein rotation of the valve gate 157 determines the flow rate of the gas from the inlet port 120 to the outlet port 130. The valve body 154 and the valve gate 157 may include optical or magnetic sensors to enable sensing of the precise position of the valve gate 157 during operation. Each of these sensors may also be arranged in communication with the EPS32 microcontroller module 140 to provide feedback on the positioning of the valve gate 157 after the servo motor 152 has been actuated through output control signals generated by the microprocessor 142 of the EPS32 microcontroller module 140.

The exhaled air from the patient may be directed back from the patient interface (gas mask or endotracheal tube) into an exhalation inlet port 195 disposed in the housing 110. During exhalation, the patient's exhaled breath is conducted from the exhalation inlet port 195 to an exhalation valve 197 to an exhalation outlet 199. An exhalation sensor 196 may be provided in line to generate a signal associated with the pressure and flow rate of the exhaled air. Once again, the exhalation sensor 196 may also communicate with the EPS32 controller module 140 and provide feedback from the exhaled air sensor 196 which may be processed by the microprocessor 142 in accordance with executable instructions saved on the memory device 145 to generate control signals for controlling the operation of the valve assembly 150. The exhaled air may then be directed out of the ventilator unit 100 through an exhalation outlet port 199. The outlet port 193 may be coupled to an exhaust unit 350 and a filtering unit 360. In some embodiments, outlet ports 199 for a plurality of interconnected ventilators 100 may be connected to a common exhaust unit 350 and a common filtering unit to ensure that any exhaled air is thoroughly disinfected. In other embodiments, smaller exhaust units and filtering units may be coupled with each ventilator unit 100. The flow of exhaust gas may also be controlled via the rotary valve 150 by coupling an exhaust inlet port 151 on the valve 150 with the exhalation valve 197 to control flow of exhaust air from the exhalation valve 197 to the outlet port 199 via an exhaust outlet port 159 provided on the valve 150 and may be controlled via the valve gate 157 such that during operation of the valve 150 when the valve gate 157 is allowing exhaled air to flow out, no air is supplied to the patient via the outlet port 158.

Referring to FIG. 3, the peripheral interface 144 for the EPS32 microcontroller module 140 may receive signals from a user to operate the ventilator 100 from a remote location by sending operational control signals from a remote computing device such as but not limited to a mobile smart phone or tablet via a wireless network using the trans-receiver 145 of the EPS32 controller module 140. Similarly, the pressure values sensed by the airway pressure sensor 160, the ambient pressure sensor 165, the airway pressure sensor 170 and the oxygen sensor 184 may be processed by the microprocessor 142 and communicated to the remote computing device. The information related to respiratory performance of the patient supported by every ventilator 100 may be continually logged to a database for offline processing and raising an alarm if any preset parameters are not satisfied during the processing. Such a system would also assist with analyzing progression of the patients breathing capability and record any observations associated with the patient's breathing behaviour.

The aforementioned configuration may be used for allowing Health professionals can interface to the unit via a web page locally hosted on the ventilator unit 100, or via a centralised control/monitoring room, or remotely via the internet. Access to the ventilator 100 control interface may include a secure interface to restrict operation and control to one or more approved operators, or health professionals with appropriate credentials.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1. A ventilator assembly comprising:
a housing;
a gas inlet port disposed in the housing and adapted to be coupled to a gas source to receive a flow of gas;
a valve assembly fluidly coupled with the gas inlet port for controlling flow of gas from the gas inlet port to a gas outlet port disposed in the housing, the gas outlet port being adapted for being coupled to a patient interface to fluidly couple the gas outlet port to the airway of a patient;
a controller module comprising a controller operatively coupled with the valve assembly to control operation of the valve assembly;
an airway pressure sensor positioned between the valve assembly and the patient interface to measure air flow output flowing into the airway of the patient;
wherein the pressure sensor is operatively connected to said controller module to control the operation of the valve assembly in response to changes in air flow output measured by the airway pressure sensor during use.

2. A ventilator assembly in accordance with claim 1 further comprising an inlet pressure sensor disposed between the gas inlet port and the valve assembly for measuring gas flow input flowing into the gas inlet port wherein the inlet pressure sensor is operatively connected to the controller module to control the operation of the valve assembly in response to changes in air flow input measured by the inlet pressure sensor during use.

3. A ventilator in accordance with claim 1 or claim 2 further comprising an oxygen inlet port adapted to be coupled to an oxygen gas source for allowing oxygen gas to flow from the oxygen gas source to the outlet port.

4. A ventilator in accordance with claim 3 further comprising an oxygen sensor for measuring flow rate of oxygen flowing into the outlet port for being conveyed to the airway of the patient.

5. A ventilator in accordance with claim 4 wherein the oxygen sensor is operatively connected to the controller module to control the operation of an oxygen regulating module in response to changes in air flow input or air flow output measured by the inlet pressure sensor or the airway pressure sensor during use.

6. A ventilator in accordance with any one of the preceding claims further comprising a relief valve disposed in the housing and adapted to be coupled with the gas outlet port for releasing gas from said relief valve when pressure of gas flowing from the valve assembly to the outlet exceeds a pre-set pressure value.

7. A ventilator in accordance with any one of the preceding claims wherein the valve assembly comprises a rotary valve assembly further comprising:
a valve body defining at least one input port and at least one output port, each port providing a separate fluid communication path between an outer surface of the valve body;
a bore extending along a longitudinal axis defined by the valve body, and
a valve gate rotatably positioned in the bore to rotate between an open position and a closed position wherein in the open position the gas is conveyed from the inlet port to the outlet port.

8. A ventilator in accordance with claim 7 wherein the valve gate comprises one or more flow passages for conveying fluid from the inlet port to the outlet port wherein rotation of the valve gate determines the flow rate of the gas from the inlet port to the outlet port.

9. A ventilator in accordance with claim 7 or claim 8 wherein the valve gate is driven by a servo motor, the servo motor being operatively coupled to the controller module for controlling the rotation of the valve gate in response to changes in air flow output measured by the airway pressure sensor and/or changes in air flow input measured by the inlet pressure sensor.

10. A ventilator in accordance with any one of the preceding claims further comprising an ambient pressure sensor for sensing ambient pressure in the vicinity of the housing, the ambient pressure being operatively coupled with the controller module to control the operation of the valve assembly in response to changes in air flow input measured by the inlet pressure sensor during use.

11. A ventilator in accordance with any one of the preceding claims wherein the controller module comprises a microprocessor arranged to be in communication with a peripheral interface wherein the peripheral interface is adapted to be connected to the airway pressure sensor.

12. A ventilator in accordance with claim 11 when dependent upon claim 2 wherein the peripheral interface is adapted to be connected to the inlet pressure sensor.

13. A ventilator in accordance with claim 11 when dependent upon claim 4 or claim 12 wherein the peripheral interface is adapted to be connected to the oxygen sensor.

14. A ventilator in accordance with any one of claims 11 to 13 wherein the microprocessor is in communication with a memory device, storing executable instructions wherein the processor, when executing said instructions to monitor one or more of said inputs received on the peripheral interface for checking if one or more predetermined criteria have been satisfied, generates control signals for operating the valve assembly in response to checking if the said one or more predetermined criteria have been satisfied.

15. A ventilator in accordance with claims 11 to 14 wherein the microprocessor is in communication with a transmitting module for transmitting one or more signals from the microprocessor, over a network, to one or more computing devices.

16. A ventilator in accordance with claims 11 to 15 wherein the processor is in communication with a receiver for receiving signals associated with instructions for operating the ventilator.

17. A ventilator in accordance with any one of claims 1 to 16 wherein the inlet port is adapted to be coupled with a source of compressed air generated by a compressor in a first operable configuration, said source of compressed air being fluidly coupled with a plurality of said inlet ports associated with corresponding ventilators simultaneously said first operating configuration.

18. A ventilator in accordance with any one of claims 1 to 17 wherein the inlet port is adapted to be coupled with a portable blower device for receiving a supply of gas from the blower

19. A system for ventilating a plurality of patients simultaneously, the system comprising: a plurality of the ventilators in accordance with any one of the preceding claims wherein the inlet port for each of the plurality of ventilators is fluidly coupled to a common gas source to receive the flow of gas and wherein the outlet port for each of the plurality of ventilators is fluidly coupled to a common exhaust.

20. A system in accordance with claim 19 further comprising an inlet gas regulator positioned in line with the common gas source for regulating the flow of gas to each of the inlet ports fluidly coupled to the common gas source.
